(19) United States
(12) Patent Application Publication
(10) Pub. No.: US 2008/0234535 A1
(43) Pub. Date: Sep. 25, 2008

(54) Device and method for thrombosis and pulmonary embolism

(75) Inventors: Henryk Malak, Ellicott City, MD (US); Barry M. Hart, Ellicott City, MD (US)

Correspondence Address: Henryk Malak, 8444 High Ridge Road, Ellicott City, MD, 21043, US

(73) Assignee: American Environmental Systems, Inc

(21) Appl. No.: 11/804,759

(22) Filed: May 21, 2007

Related U.S. Application Data
(60) Provisional application No. 60/919,531, filed on Mar. 23, 2007.

(51) Int. Cl. A61N 2/02

(52) U.S. Cl. 600/15; 601/2; 607/3

(57) ABSTRACT

This disclosure describes novel therapeutic devices and methods for thrombosis or pulmonary embolism at physiological temperature that are based upon electromagnetic pulsed energy sources of Near Infrared (NIR) and Far Infrared (FIR) that pulsed radiations are exposed to the human body for prevention and treatment. The pulsed sources may work in the devices in coincidental, uncoincidental or sequential modes with variable pulse durations, variable pulse repetitions or variable pulse intensities, and the modes or pulse properties are selected accordingly to thrombosis or pulmonary embolism conditions.

### CROSS REFERENCE TO RELATED PATENT APPLICATION

This application claims the benefit of U.S. Provisional Application Ser. No. 60/919,531 filed Mar. 23, 2007, entitled “DVT Monitoring and Therapy Device”, which is incorporated by reference.

### FIELD OF THE INVENTION

This invention relates to therapeutic medical devices and methods, and, in particular, to devices and methods for identifying, monitoring and preventing deep vein thrombosis (DVT) and pulmonary embolism (PE) by using photodynamic engineering.

### BACKGROUND OF THE INVENTION

Many people affected by thrombosis and pulmonary embolisms perceive a need for therapeutic and personal care devices with new technical advances that may enhance therapeutic treatments or provide novel therapeutic effects.

A form of venous thromboembolic disease known as Deep-vein thrombosis (also known as deep-venous thrombosis or DVT and colloquially as “economy class syndrome” do to long flights and little leg movement) is the formation of a blood clot (“thrombus”) in a deep vein. It commonly affects the leg veins, such as the great saphenous vein, the deep femoral vein, the popliteal vein, the deep plantar venous arch and other tributaries associated with these veins and flowing blood supplies back into the pelvic girdle and eventually to the heart and lungs. Occasionally the veins of the arm are affected (one especially known diagnosis is Paget-Schrötter disease). Thrombophlebitis is the more general class of pathologies of this kind. When DVT is not diagnosed early or not at all, the result may lead to pulmonary embolism, and the patient becomes a high risk for PE which can be fatal. Deep vein thrombosis and pulmonary embolism constitute major health problems throughout the world, especially after orthopedic surgery in the lower extremities. In 2003, the Centers for Disease Control and Prevention recorded in their National Discharge Surveys of Orthopedic Surgeons demonstrated approximately 364,000 hip replacements and about 451,000 knee replacements. Without the intervention of prophylactic antithrombotic drugs, anticoagulants and antiplatelet agents 80 percent of the patients would have this condition. Each year, over 2,000,000 Americans suffer from a thrombosis or embolism condition resulting in the deaths of approximately 60,000 patients.

Currently, there is only one external non-invasive medical product on the market to help minimize or prevent the reoccurrence of DVT's. These products are known as compression stockings. These stockings come in two varieties (designs) depending on how the physician wants to treat the patient and for which prognosis result is best suited for the condition. The first goal is generally to relieve the patient of associated symptoms of DVT, especially the swelling and pain. The second goal is treated is as a preventive measure known as postthrombotic syndrome.

The principle behind the compression stocking is simple and is known as graduated compression. This compression begins at the foot and ankle with a tight fit and eventually and gradually expands to a looser fit just above the knee. Medically it is designed to increase the blood flow and circulation to the upper extremities which in theory reduces the chances of de-oxygenated blood and particles within the vein to coagulate, producing a thrombus in the lower and upper legs and eventually breaking off into emboli.

Many physicians and medical professionals have questioned this principle for treating DVT conditions. Some disadvantages to this product are issues concerning patients with Congestive Heart Failure and the associated conditions sever edema to their feet and ankles, Diabetic Patients with poor circulation to the extremities such as to the metatarsus and phalanges (toes) of the foot. Diabetic Patients who have wound healing complications and who are on certain medications have difficulty with wearing these devices. The list goes on and we still have not mentioned one of the most obvious complaints and that is over time wearing these stockings become extremely uncomfortable and hot.

### SUMMARY OF THE INVENTION

By using a unique device and method that implementing electromagnetic energy pulsed sources representing two specific groups of bandwidths, one associated with Near Infrared (NIR) electromagnetic energy and the other with Far Infrared (FIR) electromagnetic energy, a device can be created to monitor, identify, treat and prevent the development of thrombosis in the extremities of the human body substantially at physiological temperature. This said device will be integrated into a non-allergenic sleeve that can be worn flat or in contour with the body such as around the leg, arm or chest and secured using hook and latch type products.

When the NIR electromagnetic energy is generated at a bandwidth ranging from 600 nm (nanometers) to 1100 nm, a pulsed electromagnetic energy will be introduced to the area presented for prevention and treatment of thrombosis and pulmonary embolism. This bandwidth will also allow the device to introduce near infrared spectroscopy (NIRS) tools to diagnose and monitor thrombosis and pulmonary embolism conditions in the body. Typical applications include biochemical and medical diagnostics (including blood sugar and oximetry). This gives the device a source for monitoring a thrombus or emboli within a vein. NIRS is based on molecular overtone and combination vibrations. Historically, such transitions are forbidden by the selection rules of quantum mechanics. As a result, the molecular absorptivity in the near IR region is typically quite small. Our research has shown one advantage is that NIR can typically penetrate much farther into a sample (such as through muscle tissues) than mid-infrared radiation. Near infrared spectroscopy is therefore not a particularly sensitive technique, but it can be very useful in probing the body with little or no sample preparation.

Another aspect of this invention uses FIR electromagnetic energy generated at a bandwidth of 8000 nm to 12000 nm. This range of electromagnetic energy is in radiation resonance with tissue, deposits its energy in deep tissue that helps to increase blood flow and improves sphygmomanometer readings in the lower extremities. This allows for continued treatment during short periods without any pneumatic or electrical stimulation of the muscle which have been mentioned in previous patents.

Electromagnetic energies of NIR and FIR can be introduced to the patient in multiple modes (settings). Based on the diagnosis and prognosis presented by the physician, one or both of the electromagnetic energies can be activated through software programming once the device is placed on the body. Modes of operation will produce a coincidental, non-coincidental and/or sequential time correlation between pulses of the electromagnetic energies when wearing the device. It will contain its own internal power source and will also be able to connect to an external power source if needed. The device is designed to be portable or stationary depending on the treatment requirements.

The design includes also a thermal insulator being placed between the patient target area and the device to maintain temperature of the irradiated body near physiological temperature. While in use the device is designed to prevent temperatures to exceed 5° C. above the physiological temperature and as well also allows lowering the body temperature accordingly to thrombosis or pulmonary embolism conditions.

In addition to this insulator, several biofeedback sensors will be placed within the design of the device allowing for continuous monitoring of medical information required to treat or prevent thrombosis or pulmonary embolism.

The device is also designed to work with an agent placed in or on the body for additional diagnosis, prevention or treatment.

### BRIEF DESCRIPTION OF THE DRAWINGS

The accompanying drawings, which are incorporated in, and constitute a part of the specification, illustrate or exemplify embodiments of the present invention and, together with the description, generally explain the principles and features of the present invention.

FIG. 1 illustrates a portable device with a speckle imaging feedback in accordance with an embodiment of the present invention.

FIG. 2 illustrates a portable device with an oximetry feedback in accordance with an embodiment of the present invention.

FIG. 3 illustrates a side view of a portable device with a thermal insulator in accordance with another embodiment of the present invention.

### DESCRIPTION OF PREFERRED EMBODIMENTS

All patents, patent applications, and literatures cited or referenced in this description are incorporated herein by reference in their entireties. In the case of inconsistencies, the present disclosure, including definitions and usage, will control.

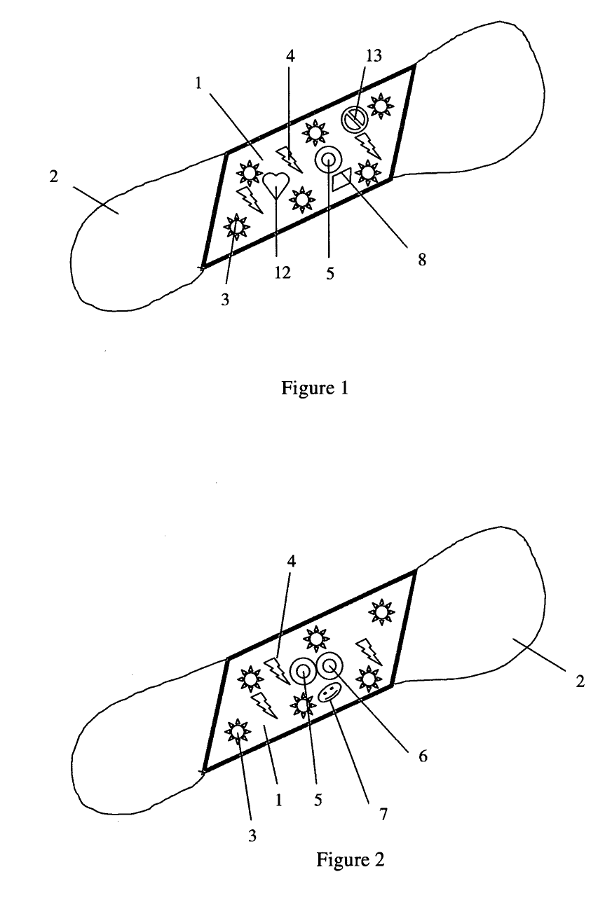

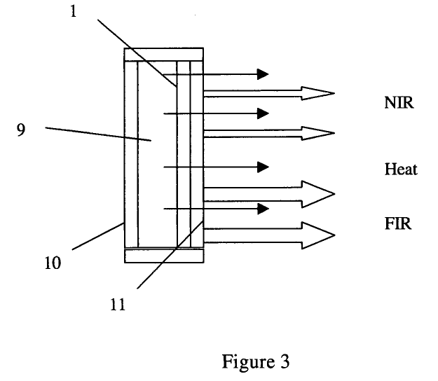

This disclosure generally describes novel therapeutic or personal care devices and methods that are based upon electromagnetic pulsed energy sources of Near Infrared (NIR) and Far Infrared (FIR) exposed to the human body for prevention or treatment thrombosis or pulmonary embolism. As denoted in the FIGS. 1 and 2, embodiments of the present invention consist of at least the both energy sources NIR 3 and FIR 4 that can be further supported by another type of pulsed energy source or a plurality of pulsed energy sources selected from the group consisting of: acoustic 13 or electric/electrostatic 12. The use of the pulsed energy sources in the devices allows maintaining substantially physiological temperature of the irradiated body during the prevention or the treatment. The sources may work in the devices in coincidental, uncoincidental or sequential modes with variable pulse durations, variable pulse repetitions or variable pulse intensities, and the modes or pulse properties are selected accordingly to thrombosis or pulmonary embolism conditions.

It should be understood that the FIGS. 1,2 and 3 illustrate certain components (i.e., PC board 1, a body strap 2, electronics 9, and a thermal insulator 11) as contained within housing 10. In alternative embodiments, however, these components may be partially or wholly external to the particular devices.

In an embodiment, the invention considers the use of the electromagnetic energy source as a single electromagnetic source or a plurality of electromagnetic sources selected from the group consisting of: light emitting diodes, super luminescence diode, organic luminescence diode, laser diode, vertical cavity laser, laser, black body radiation, or FIR luminescence materials or FIR luminescence devices.

Furthermore, devices within the scope of the present invention can be provided with an additional feature or features that can be essential for their effective performance and ease of use. Such features can be: a computer or processor to run electronics and programs for therapy, and diagnostic needs; biomedical and instrumental feedbacks with biomedical diagnostic sensors (laser 5, diode 6, photodiode 7, CCD camera 8) controlled by the computer or processor and associated electronics; a battery or AC/DC power supply; a housing with transparent or semitransparent properties to some energy sources; a data/control local and remote communication medium; and other features known in the art.

The scope of the invention also includes the use of the therapeutic devices with the hygienic or therapeutical agents whose hygienic-therapeutical properties may be enhanced by radiation of the energy source and/or the energy sources of the therapeutic devices. Such substances may enhance in the body blood flow, stimulate muscle, dissolve blood cloth, kill bacteria/viruses, bioactivate of tissue, heal wounds, and regenerate nerves, but are not limited to them. These substances can be applied externally or internally to the body.

In another embodiment, a device is further provided with a thermal insulator 11 to maintain desired thermal conditions of the irradiated body and to enhance performance of the device. Even the device without the thermal insulator will provide prevention or treatment of the body substantially at physiological temperature due to use of the pulsed energy sources, but the thermal insulator 11 is capable to change temperature of the irradiated body according to prevention or treatment needs. It is expected that the thermal insulator will maintain the body temperature within a −5 C to +5 C range of the physiological temperature, but other temperature ranges also are considered.

A person of ordinary skill in the art will appreciate the optional use of the device as a biomedical diagnostic device. Accordingly, biomedical sensors implemented into the device may provide a quantitative biofeedback from the irradiated body, which then can be used for biomedical diagnostics. FIGS. 1 and 2 illustrate examples of the placement of photon detectors 7 and 8, laser 5, and light emitting diode 6 in the portable device.

An operational mode of the pulsed energy sources in the device can be coincidental, uncoincidental or sequential or random. The energy sources may work in variable time with variable pulse durations, variable pulse intensities and variable frequencies. Selection of the mode may depend on the conditions of the treated body, and the mode or modes can be executed in the device manually or automatically with pre-programmed options.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. A device for thrombosis/pulmonary embolism comprising of: an electromagnetic composition capable to prevent or treat of thrombosis/pulmonary embolism in a body at physiological temperature or within a 5 C range of physiological temperature, the composition comprising a pulsed NIR electromagnetic source and a pulsed FIR electromagnetic source, the both NIR and FIR sources radiate pulses that are time correlated or property correlated to each other for the treatment or the prevention purposes; and an electronic circuit to administrate the composition accordingly to the treatment or the prevention needs.

2. The device of claim 1, wherein the composition radiates electromagnetic energy within spectral regions of: 600 nm to 1100 nm and 8000 nm to 12000 nm.

3. The device of claim 1, wherein the composition is a single type electromagnetic source or a plurality of electromagnetic sources.

4. The device of claim 1, wherein the property correlation between the pulses in the composition is related to a pulse duration, a pulse repetition or pulse intensity.

5. The device of claim 1, wherein the time correlation between the pulses in the composition is a coincidental, an uncoincidental or a sequential.

6. The device of claim 1, wherein the device further comprising of a thermal insulator placed between the device and the body and the thermal insulator is capable to change temperature of the body accordingly to thrombosis/pulmonary embolism treatment or prevention needs.

7. The device of claim 1, wherein the device further comprising a feedback sensor or a plurality of feedback sensors for diagnosing and monitoring of the thrombosis/pulmonary embolism conditions in the body.

8. The device of claim 1, wherein the device is capable of working with an external agent placed in/on the body for diagnostic or prevention/treatment purposes.

9. The device of claim 1, wherein the composition is further supported by another pulsed energy source or a plurality pulsed energy sources selected from the group of: acoustic, electric, electrostatic, or magnetic.

10. The device of claim 1, wherein the device is a portable device or a stationary device.

11. A method for preventing or treating thrombosis/pulmonary embolism comprising steps of: providing an electromagnetic composition capable of a prevention or a treatment of thrombosis or pulmonary embolism in a body at physiological temperature or within a 5 C range of physiological temperature, the composition comprising a pulsed NIR electromagnetic radiation and a pulsed FIR electromagnetic radiation, the both NIR and FIR radiate pulses that are time correlated or property correlated to each other for the treatment or the prevention purposes, and the pulses of the NIR electromagnetic radiation induces a different type of a reaction in the irradiated body than the pulses of the FIR electromagnetic radiation for the treatment or the prevention purposes; allowing the body to be irradiated by the composition; and providing electronic means to administrate the composition accordingly to a thrombosis/pulmonary embolism treatment or prevention needs.

12. The method of claim 11, wherein the composition radiates electromagnetic energy within spectral regions of: 600 nm to 1100 nm and 8000 nm to 12000 nm, and the radiation wavelengths of the composition are selected accordingly to thrombosis/pulmonary embolism conditions of the body.

13. The method of claim 11, wherein the property correlation between the pulses in the composition is related to a pulse duration, a pulse repetition or pulse intensity.

14. The method of claim 11, wherein the time correlation between the pulses in the composition is a coincidental, an uncoincidental or a sequential.

15. The method of claim 11, wherein the method further comprising step of: providing means to change temperature of the body accordingly to thrombosis/pulmonary embolism conditions of the body

16. The method of claim 11, wherein the method is further optimized and controlled by a feedback.

17. The method of claim 11, wherein the method is further extended to the body containing an external agent.

18. The method of claim 11, wherein the composition is further supported by another pulsed energy or a plurality pulsed energy sources selected from the group of: acoustic, electric, electrostatic, or magnetic.
